(19) United States
(12) Patent Application Publication
(10) Pub. No.: US 2012/0264131 A1
(43) Pub. Date: Oct. 18, 2012

(54) CHANGES IN THE EXPRESSION OF miR-200c/141 CLUSTER OF microRNAs AS BIOMARKERS FOR EPITHELIAL-TO-MESENCHYMAL TRANSITION IN HUMAN COLORECTAL CANCER METASTASIS

(75) Inventors: Ajay Goel, Dallas, TX (US); C. Richard Boland, Dallas, TX (US); Keun Hur, Dallas, TX (US)

(73) Assignee: BAYLOR RESEARCH INSTITUTE, Dallas, TX (US)

(21) Appl. No.: 13/408,836

(22) Filed: Feb. 29, 2012

Related U.S. Application Data
(60) Provisional application No. 61/454,053, filed on Mar. 18, 2011.

(51) Int. Cl. C12Q 1/68; C07H 21/02

(52) U.S. Cl. 435/6.12; 435/6.14; 536/23.1

(57) ABSTRACT

The present invention includes methods, kits and biomarkers for detecting and determining the development of colorectal cancer (CRC) metastasis based on changes in the expression pattern of one or more microRNAs (miR) or miR clusters that include the miR-200/141 family.

### STATEMENT OF FEDERALLY FUNDED RESEARCH

This invention was made with U.S. Government support under Contract Nos. R01 CA72851 and CA129286 awarded by the National Cancer Institute (NCI)/National Institutes of Health (NIH). The government has certain rights in this invention.

### CROSS-REFERENCE TO RELATED APPLICATIONS

None.

### TECHNICAL FIELD OF THE INVENTION

The present invention relates in general to the field of cancer detection, and more particularly, to methods for monitoring changes in expression of the miR-200 family of microRNAs and its role in the colorectal cancer metastasis development.

### INCORPORATION-BY-REFERENCE OF MATERIALS FILED ON COMPACT DISC

None.

### REFERENCE TO A SEQUENCE LISTING

None.

### BACKGROUND OF THE INVENTION

Without limiting the scope of the invention, its background is described in connection with biomarkers for cancer metastasis development.

U.S. Patent Application No. 20100317533 (Lou et al. 2010) provides a panel of biomarkers of tumour metastasis comprising any two of carbonic anhydrase-9 (CAIX), vascular endothelial growth factor C (VEGF-C), ephrin A5 (EFNA5), eph receptor B2 (EPHB2), transforming growth factor beta 3 (TGF-β3), pyruvate dehydrogenase kinase isoenzyme-3 (PDK3), carbonic anhydrase-12 (CAXII), keratin 14 (KRT14), hypoxia inducible factor 1 alpha subunit (HIF-1α), or tenascin C (TNC). CAIX, VEGF-C, EFNA5, EPHB2, TGF-β3 or PDK3 may be indicators of moderate metastatic potential, while CAXII, KRT14, HIF-1α, or TNC may be indicators of high metastatic potential. There is also provided a method of determining risk of tumour metastasis using the aforementioned biomarkers is also provided. The biomarkers may be used in diagnosis, prognosis, treatment selection, or to test putative therapeutics. The biomarkers may be used to assess malignancies or cancers having hypoxic regions, such as breast cancer.

U.S. Patent Application No.20100120898 (Croce et al. 2010) discloses methods and compositions for the diagnosis, prognosis and treatment of Hepatocellular carcinoma (HCC). Also provided are methods of identifying anti-HCC agents. The Croce invention provides a method diagnosing whether a subject has, or is at risk for developing, hepatocellular carcinoma (HCC), comprising measuring the level of at least one miR gene product in a test sample from the subject, wherein an alteration in the level of the miR gene product in the test sample, relative to the level of a corresponding miR gene product in a control sample, is indicative of the subject either having, or being at risk for developing, HCC.

### SUMMARY OF THE INVENTION

The present inventors demonstrate the role of miR-200 family members (miR-200b, miR-200c, miR-141 and miR-429) in colorectal cancer (CRC) metastasis development.

In one embodiment the instant invention provides a method for diagnosing or detecting pre-cancer, colorectal cancer (CRC) tumor progression, or metastasis in a human subject comprising the steps of: obtaining one or more biological samples from the subject, wherein the biological samples are selected from the group consisting of a tissue sample, a fecal sample, a cell homogenate, one or more biological fluids, or any combinations thereof, measuring an overall expression pattern or level of one or more microRNAs (miR) or miR clusters in one or more cells obtained from the biological samples of the subject, and comparing the overall expression pattern of the one or more miR or miR clusters from the biological sample of the subject suspected of suffering from colorectal cancer with the overall expression pattern of the one or more miR or miR clusters from a biological sample of a normal subject, wherein the normal subject is a healthy subject not suffering from colorectal cancer, wherein a change in the overall expression pattern of the one or more miR or miR clusters in the biological sample of the subject is indicative of CRC tumor progression, metastasis or both.

In one aspect of the method disclosed hereinabove the biological samples are selected from the group consisting of a tissue sample, a fecal sample, a cell homogenate, a blood sample, one or more biological fluids, or any combinations thereof. In another aspect one or more miR comprise microRNAs from the miR-200 family, wherein the miR-200 family comprises miR-200b, miR-200a, miR-200c, miR-141, and miR-429. In yet another aspect the one or more miR clusters comprise miR200c/141 cluster, miR200b, a/429 cluster, or both.

In a related aspect a significant decrease in the expression levels of miR-200c, miR-141, miR-200c/141 cluster or any combinations thereof is indicative of CRC tumor progression. In another aspect a significant increase in the expression levels of miR-200c, miR-141, miR-200c/141 cluster or any combinations thereof is indicative of liver metastasis. In a specific aspect the expression level of the one or more miR or miR clusters is measured by quantitative real-time PCR. In another aspect the method is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.

Another embodiment of the present invention relates to a biomarker for colorectal cancer disease progression, metastasis or both wherein the biomarker comprises one or more microRNAs (miR) or miR clusters and a change in the overall expression of the one or more miR, miR clusters or both in colorectal cancer cells obtained from a patient is indicative of colorectal cancer disease progression, metastasis or both when compared to the overall expression of the one or more miR, miR clusters or both expression in normal colorectal cancer cells or colorectal cancer cells obtained at an earlier timepoint from the same patient. In one aspect of the biomarker described hereinabove the one or more miR comprise microRNAs from the miR-200 family, wherein the miR-200 family comprises miR-200b, miR-200a, miR-200c, miR-141, and miR-429. In another aspect the one or more miR clusters comprise miR200c/141 cluster, miR200b, a/429 cluster, or both. In yet another aspect a significant decrease in the expression levels of miR-200c, miR-141, miR-200c/141 cluster or any combinations thereof is indicative of CRC tumor progression. In another aspect a significant increase in the expression levels of miR-200c, miR-141, miR-200c/141 cluster or any combinations thereof is indicative of liver metastasis.

In yet another embodiment the present invention discloses a biomarker for colorectal cancer (CRC) disease progression, metastasis or both wherein the biomarker comprises miR-200c, miR-141, miR-200c/141 cluster or any combinations thereof and a change in the overall expression of the miR-200c, miR-141, or miR-200c/141 cluster in colorectal cancer cells obtained from a patient is indicative of colorectal cancer disease progression, metastasis or both when compared to the overall expression of the miR-200c, miR-141, or miR-200c/141 cluster expression in normal CRC cells or colorectal cancer cells obtained at an earlier timepoint from the same patient. In one aspect of the biomarker described above a significant decrease in the expression levels of miR-200c, miR-141, miR-200c/141 cluster or any combinations thereof is indicative of CRC tumor progression. In another aspect a significant increase in the expression levels of miR-200c, miR-141, miR-200c/141 cluster or any combinations thereof is indicative of liver metastasis.

The present invention also discloses a kit for a diagnosis of colorectal cancer (CRC) comprising: biomarker detecting reagents for determining a differential expression level of miR-200c, miR-141, miR-200c/141 cluster or any combinations thereof and instructions for their use in diagnosing risk for colorectal cancer, wherein the instruction comprise step-by-step directions to compare the expression level of miR-200c, miR-141, miR-200c/141 cluster or any combinations thereof from one or more samples obtained from a subject suspected of having colorectal cancer with the expression level of miR-200c, miR-141, miR-200c/141 cluster or any combinations thereof in one or more sample from a normal subject, wherein the normal subject is a healthy subject not suffering from colorectal cancer.

In one aspect of the kit disclosed above the samples are selected from the group consisting of a tissue sample, a fecal sample, a cell homogenate, a blood sample, one or more biological fluids, or any combinations thereof. In another aspect of the kit disclosed herein a significant decrease in the expression levels of miR-200c, miR-141, miR-200c/141 cluster or any combinations thereof is indicative of CRC tumor progression. In yet another aspect a significant increase in the expression levels of miR-200c, miR-141, miR-200c/141 cluster or any combinations thereof is indicative of liver metastasis.

The present invention in one embodiment provides a method for selecting a cancer therapy for a patient diagnosed with colorectal cancer, the method comprising: determining an overall expression level of miR-200c, miR-141, miR-200c/141 cluster or any combinations thereof from a biological sample of the patient to determine a CRC disease progression, metastasis or both; and selecting the cancer therapy based on the determination of the CRC disease progression, metastasis or both in the patient.

In one aspect of the method disclosed hereinabove the biological samples are selected from the group consisting of a tissue sample, a fecal sample, a cell homogenate, a blood sample, one or more biological fluids, or any combinations thereof. In another aspect the step of determining the overall level of expression of miR-200c, miR-141, miR-200c/141 cluster or any combinations thereof comprises analyzing a tissue sample suspected of being colorectal cancer for miR-200c, miR-141, or miR-200c/141 cluster expression. In yet another aspect a significant decrease in the expression levels of miR-200c, miR-141, miR-200c/141 cluster or any combinations thereof is indicative of CRC tumor progression. In another aspect a significant increase in the expression levels of miR-200c, miR-141, miR-200c/141 cluster or any combinations thereof is indicative of liver metastasis.

One embodiment of the present invention is related to a method for stratifying a patient in a subgroup of colorectal cancer (CRC), the method comprising the steps of: determining an overall expression of miR-200c, miR-141, miR-200c/141 cluster or any combinations thereof in cells suspected of being CRC cells from the patient and predicting the stage of the CRC by checking for a significant decrease in the expression levels of miR-200c, miR-141, miR-200c/141 cluster or any combinations thereof in comparison to the expression of miR-200c, miR-141, or miR-200c/141 cluster in normal CRC cells.

In yet another embodiment the present invention provides a method of performing a clinical trial to evaluate a candidate drug believed to be useful in treating a disease state associated with changes expression of miR-200c, miR-141, miR-200c/141 cluster or any combinations thereof, the method comprising: a) measuring the level of miR-200c, miR-141 or miR-200c/141 cluster expression from tissue suspected of having colorectal cancer (CRC) from a set of patients; b) administering a candidate drug to a first subset of the patients, and a placebo to a second subset of the patients; a comparable drug to a second subset of the patients; or a drug combination of the candidate drug and another active agent to a second subset of patients; c) repeating step a) after the administration of the candidate drug or the placebo, the comparable drug or the drug combination; and d) determining if the candidate drug reduces the number of colorectal cells that have a decrease in the expression of miR-200c, miR-141, miR-200c/141 cluster or any combinations thereof that is statistically significant as compared to any reduction occurring in the second subset of patients, wherein a statistically significant reduction indicates that the candidate drug is useful in treating said disease state.

Furthermore, the present invention describes a method for diagnosing or detecting a pre-cancer, colorectal cancer (CRC), tumor progression, or metastasis in a human subject comprising the steps of: i) identifying the human subject suffering form or suspected of suffering from colorectal cancer, ii) obtaining one or more biological samples from the subject, wherein the biological samples are selected from the group consisting of a tissue sample, a fecal sample, a cell homogenate, one or more biological fluids, or any combinations thereof, iii) measuring an overall expression pattern or level of one or more microRNAs (miR) or miR clusters in one or more cells obtained from the biological samples of the subject, and iv) comparing the overall expression pattern of the one or more miR or miR clusters from the biological sample of the subject suspected of suffering from colorectal cancer with the overall expression pattern of the one or more miR or miR clusters from a biological sample of a normal subject, wherein the normal subject is a healthy subject not suffering from colorectal cancer, wherein a change in the overall expression pattern of the one or more miR or miR clusters in the biological sample of the subject is indicative of CRC tumor progression, metastasis or both.

In one aspect the biological samples are selected from the group consisting of a tissue sample, a fecal sample, a cell homogenate, a blood sample, one or more biological fluids, or any combinations thereof. In another aspect the one or more miR comprise microRNAs from the miR-200 family, wherein the miR-200 family comprises miR-200b, miR-200a, miR-200c, miR-141, and miR-429. In yet another aspect the one or more miR clusters comprise miR200c/141 cluster, miR200b, a/429 cluster, or both. In related aspects a significant decrease in the expression levels of miR-200c, miR-141, miR-200c/141 cluster or any combinations thereof is indicative of CRC tumor progression and a significant increase in the expression levels of miR-200c, miR-141, miR-200c/141 cluster or any combinations thereof is indicative of liver metastasis. In another aspect the expression level of the one or more miR or miR clusters is measured by quantitative real-time PCR. Finally, the method of the present invention as described hereinabove is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions which are characterized by cancer of cells of the duodenum and small intestine (jejunum and ileum).

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH2C12, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me2S (source: infection; diagnosis: trench mouth).

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The present invention describes the role of miR-200 family members (miR-200b, miR-200c, miR-141 and miR-429) in colorectal cancer (CRC) metastasis development and the measurement of the relation between a change in expression patterns of the miR-200 family of microRNAs and CRC metastasis and employing this change as a biomarker for detecting or predicting CRC metastasis.

The development of metastases is one of the main causes of the cancer-related death in CRC. The epithelial-to-mesenchymal transition (EMT) occurs during tumor progression, which provides cancer cells with invasive and metastatic properties. The molecular mechanisms by which colorectal cancer cells exploit the hepatic microenvironment for selective growth and survival remain obscure. Recently, loss of expression of the miR-200 family of microRNAs has been linked to an aggressive cancer phenotype in breast cancers. In this context, miR-200 families of miRNAs are proposed to inhibit EMT process by targeting the E-cadherin transcriptional repressors, ZEB1 and ZEB2. Although EMT plays a central role in metastasis, the contribution of miR-200 family members in the development of distant metastasis in CRC remains unclear.

The present inventors analyzed a panel of CRC cell lines with different metastatic potential (HCT116, SW480 and SW620), as well as clinical specimens from 55 patients with primary CRC and matched liver metastasis tissues. MicroRNAs expression of miR-200b, miR-200c, miR-141 and miR-429 was determined by quantitative real-time PCR and the data were normalized relative to miR-16 expression.

Lower levels of miR-200c/141 cluster expression were observed in SW620 (derived from lymph node metastasis) compared to HCT116 and SW480 cell lines (derived from primary CRCs), but no significant changes in miR-200b/429 expression were observed in any of the cell lines. When the inventors analyzed miR-200 family expression levels in clinical specimens, the relative levels of miR-200c expression were progressively lower with higher tumor stages in primary CRC tissues (P<0.05). However, miR-200c expression was significantly up-regulated in liver metastasis compared to the corresponding matched primary CRC (P<0.001). Similar observations were made for miR-141 expression in metastatic liver foci compared to the corresponding matched primary CRC.

This study provides novel insights into the involvement of miR-200/141 family members in the development of CRC metastasis. The decreased expression of the miR-200/141 cluster in primary CRCs supports the participation of these miRNAs in the EMT process. In contrast, the increased expression of the miR-200/141 cluster in liver metastasis suggests a potential role in the initiation of mesenchymal-to-epithelial transition (MET) process, in which increased expression of these miRNAs facilitates the enhanced proliferation of these metastatic tumor cells following their settlement in the liver.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20100317533: Biomarkers of Cancer Metastasis.

U.S. Patent Application No.20100120898: MicroRNA Expression Signature for Predicting Survival and Metastases in Hepatocellular Carcinoma.

What is claimed is:

1. A method for diagnosing or detecting pre-cancer, colorectal cancer (CRC) tumor progression, or metastasis in a human subject comprising the steps of:
obtaining one or more biological samples from the subject, wherein the biological samples are selected from the group consisting of a tissue sample, a fecal sample, a cell homogenate, one or more biological fluids, or any combinations thereof;
measuring an overall expression pattern or level of one or more microRNAs (miR) or miR clusters in one or more cells obtained from the biological samples of the subject; and
comparing the overall expression pattern of the one or more miR or miR clusters from the biological sample of the subject suspected of suffering from colorectal cancer with the overall expression pattern of the one or more miR or miR clusters from a biological sample of a normal subject, wherein the normal subject is a healthy subject not suffering from colorectal cancer, wherein a change in the overall expression pattern of the one or more miR or miR clusters in the biological sample of the subject is indicative of CRC tumor progression, metastasis or both.

2. The method of claim 1, wherein the biological samples are selected from the group consisting of a tissue sample, a fecal sample, a cell homogenate, a blood sample, one or more biological fluids, or any combinations thereof.

3. The method of claim 1, wherein the one or more miR comprise microRNAs from the miR-200 family, wherein the miR-200 family comprises miR-200b, miR-200a, miR-200c, miR-141, and miR-429.

4. The method of claim 1, wherein the one or more miR clusters comprise miR200c/141 cluster, miR200b, a/429 cluster, or both.

5. The method of claim 1, wherein a significant decrease in the expression levels of miR-200c, miR-141, miR-200c/141 cluster or any combinations thereof is indicative of CRC tumor progression.

6. The method of claim 1, wherein a significant increase in the expression levels of miR-200c, miR-141, miR-200c/141 cluster or any combinations thereof is indicative of liver metastasis.

7. The method of claim 1, wherein the expression level of the one or more miR or miR clusters is measured by quantitative real-time PCR.

8. The method of claim 1, wherein the method is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.

9. A biomarker for colorectal cancer disease progression, metastasis or both wherein the biomarker comprises one or more microRNAs (miR) or miR clusters and a change in the overall expression of the one or more miR, miR clusters or both in colorectal cancer cells obtained from a patient is indicative of colorectal cancer disease progression, metastasis or both when compared to the overall expression of the one or more miR, miR clusters or both expression in normal colorectal cancer cells or colorectal cancer cells obtained at an earlier timepoint from the same patient.

10. The biomarker of claim 9, wherein the one or more miR comprise microRNAs from the miR-200 family, wherein the miR-200 family comprises miR-200b, miR-200a, miR-200c, miR-141, and miR-429.

11. The biomarker of claim 9, wherein the one or more miR clusters comprise miR200c/141 cluster, miR200b, a/429 cluster, or both.

12. The biomarker of claim 9, wherein a significant decrease in the expression levels of miR-200c, miR-141, miR-200c/141 cluster or any combinations thereof is indicative of CRC tumor progression.

13. The biomarker of claim 9, wherein a significant increase in the expression levels of miR-200c, miR-141, miR-200c/141 cluster or any combinations thereof is indicative of liver metastasis.

14. A biomarker for colorectal cancer (CRC) disease progression, metastasis or both wherein the biomarker comprises miR-200c, miR-141, miR-200c/141 cluster or any combinations thereof and a change in the overall expression of the miR-200c, miR-141, or miR-200c/141 cluster in colorectal cancer cells obtained from a patient is indicative of colorectal cancer disease progression, metastasis or both when compared to the overall expression of the miR-200c, miR-141, or miR-200c/141 cluster expression in normal CRC cells or colorectal cancer cells obtained at an earlier timepoint from the same patient.

15. The biomarker of claim 14, wherein a significant decrease in the expression levels of miR-200c, miR-141, miR-200c/141 cluster or any combinations thereof is indicative of CRC tumor progression.

16. The biomarker of claim 14, wherein a significant increase in the expression levels of miR-200c, miR-141, miR-200c/141 cluster or any combinations thereof is indicative of liver metastasis.

17. A kit for a diagnosis of colorectal cancer (CRC) comprising:
biomarker detecting reagents for determining a differential expression level of miR-200c, miR-141, miR-200c/141 cluster or any combinations thereof; and
instructions for their use in diagnosing risk for colorectal cancer, wherein the instruction comprise step-by-step directions to compare the expression level of miR-200c, miR-141, miR-200c/141 cluster or any combinations thereof from one or more samples obtained from a subject suspected of having colorectal cancer with the expression level of miR-200c, miR-141, miR-200c/141 cluster or any combinations thereof in one or more sample from a normal subject, wherein the normal subject is a healthy subject not suffering from colorectal cancer.

18. The kit of claim 17, wherein the samples are selected from the group consisting of a tissue sample, a fecal sample, a cell homogenate, a blood sample, one or more biological fluids, or any combinations thereof.

19. The kit of claim 17, wherein a significant decrease in the expression levels of miR-200c, miR-141, miR-200c/141 cluster or any combinations thereof is indicative of CRC tumor progression.

20. The kit of claim 17, wherein a significant increase in the expression levels of miR-200c, miR-141, miR-200c/141 cluster or any combinations thereof is indicative of liver metastasis.

21. A method for selecting a cancer therapy for a patient diagnosed with colorectal cancer, the method comprising:
determining an overall expression level of miR-200c, miR-141, miR-200c/141 cluster or any combinations thereof from a biological sample of the patient to determine a CRC disease progression, metastasis or both; and
selecting the cancer therapy based on the determination of the CRC disease progression, metastasis or both in the patient.

22. The method of claim 21, wherein the biological samples are selected from the group consisting of a tissue sample, a fecal sample, a cell homogenate, a blood sample, one or more biological fluids, or any combinations thereof.

23. The method of claim 21, wherein the step of determining the overall level of expression of miR-200c, miR-141, miR-200c/141 cluster or any combinations thereof comprises analyzing a tissue sample suspected of being colorectal cancer for miR-200c, miR-141, or miR-200c/141 cluster expression.

24. The method of claim 21, wherein a significant decrease in the expression levels of miR-200c, miR-141, miR-200c/141 cluster or any combinations thereof is indicative of CRC tumor progression.

25. The method of claim 21, wherein a significant increase in the expression levels of miR-200c, miR-141, miR-200c/141 cluster or any combinations thereof is indicative of liver metastasis.

26. A method for stratifying a patient in a subgroup of colorectal cancer (CRC), the method comprising the steps of:
determining an overall expression of miR-200c, miR-141, miR-200c/141 cluster or any combinations thereof in cells suspected of being CRC cells from the patient; and
predicting the stage of the CRC by checking for a significant decrease in the expression levels of miR-200c, miR-141, miR-200c/141 cluster or any combinations thereof in comparison to the expression of miR-200c, miR-141, or miR-200c/141 cluster in normal CRC cells.

27. A method of performing a clinical trial to evaluate a candidate drug believed to be useful in treating a disease state associated with changes expression of miR-200c, miR-141, miR-200c/141 cluster or any combinations thereof, the method comprising:
a) measuring the level of miR-200c, miR-141 or miR-200c/141 cluster expression from tissue suspected of having colorectal cancer (CRC) from a set of patients;
b) administering a candidate drug to a first subset of the patients, and
a placebo to a second subset of the patients;
a comparable drug to a second subset of the patients; or
a drug combination of the candidate drug and another active agent to a second subset of patients;
c) repeating step a) after the administration of the candidate drug or the placebo, the comparable drug or the drug combination; and
d) determining if the candidate drug reduces the number of colorectal cells that have a decrease in the expression of miR-200c, miR-141, miR-200c/141 cluster or any combinations thereof that is statistically significant as compared to any reduction occurring in the second subset of patients, wherein a statistically significant reduction indicates that the candidate drug is useful in treating said disease state.

28. A method for diagnosing or detecting a pre-cancer, colorectal cancer (CRC), tumor progression, or metastasis in a human subject comprising the steps of:
identifying the human subject suffering form or suspected of suffering from colorectal cancer;
obtaining one or more biological samples from the subject, wherein the biological samples are selected from the group consisting of a tissue sample, a fecal sample, a cell homogenate, one or more biological fluids, or any combinations thereof;
measuring an overall expression pattern or level of one or more microRNAs (miR) or miR clusters in one or more cells obtained from the biological samples of the subject; and
comparing the overall expression pattern of the one or more miR or miR clusters from the biological sample of the subject suspected of suffering from colorectal cancer with the overall expression pattern of the one or more miR or miR clusters from a biological sample of a normal subject, wherein the normal subject is a healthy subject not suffering from colorectal cancer, wherein a change in the overall expression pattern of the one or more miR or miR clusters in the biological sample of the subject is indicative of CRC tumor progression, metastasis or both.

29. The method of claim 28, wherein the biological samples are selected from the group consisting of a tissue sample, a fecal sample, a cell homogenate, a blood sample, one or more biological fluids, or any combinations thereof.

30. The method of claim 28, wherein the one or more miR comprise microRNAs from the miR-200 family, wherein the miR-200 family comprises miR-200b, miR-200a, miR-200c, miR-141, and miR-429.

31. The method of claim 28, wherein the one or more miR clusters comprise miR200c/141 cluster, miR200b, a/429 cluster, or both.

32. The method of claim 28, wherein a significant decrease in the expression levels of miR-200c, miR-141, miR-200c/141 cluster or any combinations thereof is indicative of CRC tumor progression.

33. The method of claim 28, wherein a significant increase in the expression levels of miR-200c, miR-141, miR-200c/141 cluster or any combinations thereof is indicative of liver metastasis.

34. The method of claim 28, wherein the expression level of the one or more miR or miR clusters is measured by quantitative real-time PCR.

35. The method of claim 28, wherein the method is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.
